(19) United States
(12) Patent Application Publication
(10) Pub. No.: US 2021/0022980 A1
(43) Pub. Date: Jan. 28, 2021

(54) HAIR-PROTECTING COMPOSITION COMPRISING COLLAGEN AND MANUFACTURING METHOD THEREFOR

(71) Applicant: BEAUTEE COLLAGEN CO., LTD., Cheongju-si (KR)

(72) Inventor: Gwan Mo LEE, Cheongju-si (KR)

(21) Appl. No.: 17/066,275

(22) Filed: Oct. 8, 2020

Related U.S. Application Data
(63) Continuation of application No. PCT/KR2019/003296, filed on Mar. 21, 2019.

(30) Foreign Application Priority Data
Apr. 13, 2018 (KR) 10-2018-0043262

(51) Int. Cl. A61K 8/65; A61K 8/64; A61K 8/87; A61Q 5/00

(52) U.S. Cl. CPC A61K 8/65; A61K 8/64; A61Q 5/002; A61Q 5/00; A61K 8/87

(57) ABSTRACT

A hair-protective composition includes collagen, a polyurethane binder, sericin and water. The collagen is contained in an amount of 10 wt % to 30 wt % based on a total weight of the hair-protective composition. A method of preparing the hair-protective composition includes melting collagen in water, dissolving sericin in the melted collagen and performing filtration and performing addition with a polyurethane binder and performing stirring. The hair-protective composition including collagen can be effectively used in techniques for protecting and softening the hair in the field of synthetic hair wigs and human hair wigs.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Technical Field

The present disclosure relates to a hair-protective composition and a method of preparing the same, and more particularly to a hair-protective composition including collagen and a method of preparing the same.

### Discussion of Related Technology

Wigs are made of synthetic hair or human hair, and when a wig is used, sweat and sebum are secreted from the scalp of the person wearing the wig, and these secretions stick to the hair of the wig. Furthermore, pollutants, dust, bacteria and the like in the air also adhere to the wig. Accordingly, the flexibility and moisturized characteristic of the wig are deteriorated, the hair thereof becomes dry and stiff, and the glossiness decreases, so the aesthetic appearance thereof is deteriorated to the extent that the hair of the wig is easily distinguished from human hair.

Moreover, it is difficult to comb the wig because static electricity is readily generated in the hair and the hair is easily tangled. Human hair is generally composed of transparent scaly cells (hereinafter referred to as “cuticles”) whose outermost layer is usually called cuticles. If the cuticles corresponding to the shell of the other body skin are aligned in one direction, the hair is easily combed, but if the direction of the scales of even as little as 4% of hair is changed, the hair becomes tangled.

In addition, synthetic hair wigs have come to be widely used in recent years for the reason that human hair wigs are expensive and difficult to keep styled, but there are tangling and static electricity, similar to human hair, non-natural gloss/luster, rough hair shape, insufficient moisture absorption and the like.

Hence, there may be a need to align the cuticles of the hair and to maintain the moisturized characteristic and flexibility of human hair wigs and synthetic hair wigs. In order to address the foregoing, the outer skin of the hair may be coated with a softener, etc. during the manufacture of wigs, and wig wearers may wash wigs using shampoo for wigs, or may request wig professionals to wash wigs. However, all of these methods have only temporary coating, moisturizing or softening effects, and the lasting utility and convenient use of the wig are not ensured.

The disclosure of this section is to provide background information relating to the invention. Applicant does not admit that any information contained in this section constitutes prior art.

### SUMMARY

One aspect of the present disclosure provides a technique for protecting the hair of a wig capable of preventing static electricity and maintaining a moisturizing effect because a softener or the like, applied thereto, is not removed but remains for a long time, even after washing. Another aspect of the present disclosure provides a hair protection technique capable of imparting silky flexibility and an excellent nutrition to synthetic-hair and human-hair wigs and chemical-type fibers, and also capable of imparting superior moisture absorption capacity and a natural tactile sensation to give chemical and artificial products the same hydrophilicity and moisturized characteristic as natural products. Still another aspect of the present disclosure provides a composition of certain components including collagen, wherein the composition has the ability to promote penetration of an active ingredient, restore damaged hair, increase elasticity, reduce static electricity, and remain even after washing, and may thus be used as a hair-protective composition.

Accordingly, the present disclosure is provides a hair-protective composition including collagen and a method of preparing the same.

An aspect of the present disclosure provides a hair-protective composition including collagen, a polyurethane binder, sericin and water.

In an embodiment thereof, the collagen may be contained in an amount of 10 to 30 wt % based on the total weight of the hair-protective composition, and the polyurethane binder may be prepared by mixing 50 parts by weight of polyoxyethylene polyoxypropylene, 20 parts by weight of polyester polyol, 10 parts by weight of isophorone diisocyanate, 10 parts by weight of N-methyldiethanolamine, 5 parts by weight of MEK (methyl ethyl ketone) oxime, 3 parts by weight of a bismuth-type carboxylate catalyst, and 2 parts by weight of dimethylformamide, and may be contained in an amount of 10 to 40 wt % based on the total weight of the hair-protective composition. Also, the sericin may be contained in an amount of 1 to 10 wt % based on the total weight of the hair-protective composition.

In an embodiment thereof, the hair may be synthetic hair or human hair.

Another aspect of the present disclosure provides a method of preparing a hair-protective composition, including: (a) melting collagen in water; (b) dissolving sericin in the melted collagen and performing filtration; and (c) performing addition with a polyurethane binder and performing stirring.

According to the present disclosure, when a hair-protective composition including collagen, a polyurethane binder, sericin and water is used, the hair-protective composition is very effective at allowing collagen to penetrate to the center of the hair, restoring the surface of damaged hair, reducing the cracking of hair, increasing elasticity, and reducing the generation of static electricity, and is capable of exhibiting 50% retention of the hair-protective component, even after washing 20 to 30 times, and of increasing a moisturized characteristic.

Therefore, the hair-protective composition including collagen, a polyurethane binder, sericin and water and the method of preparing the same according to the present disclosure can be effectively used in techniques for protecting and softening the hair in the field of synthetic hair wigs or human hair wigs.

### BRIEF DESCRIPTION OF DRAWINGS

FIG. 1A shows micrographs (×3,700 and ×430) before treatment of human hair with AMICELL, and FIG. 1B shows micrographs (×3,700 and ×430) after treatment of human hair with AMICELL.

FIG. 2A shows a hair image before treatment of human hair with AMICELL, FIG. 2B shows a hair image immediately after treatment of human hair with AMICELL, and FIG. 2C shows a hair image 1 month after treatment of human hair with AMICELL.

FIG. 3A shows a micrograph before treatment of human hair with AMICELL, FIG. 3B shows a micrograph after treatment of human hair with AMICELL, and FIG. 3C shows a fluorescence image of the cross section of human hair after allowing AMICELL mixed with a fluorescent substance to penetrate the hair.

### DETAILED DESCRIPTION

Various embodiments of the present are discussed below.

Embodiments of the present disclosure pertain to a hair-protective composition including collagen, a polyurethane binder, sericin and water.

The collagen may be used without limitation, so long as it is collagen that is commonly used, and in embodiments, collagen prepared according to Korean Patent No. 10-1394230, in particular, Alaska Pollock collagen, is used. Here, the collagen may be contained in an amount of 10 to 30 wt % based on the total weight of the hair-protective composition.

The polyurethane binder may be prepared by mixing 50 parts by weight of polyoxyethylene polyoxypropylene, 20 parts by weight of polyester polyol, 10 parts by weight of isophorone diisocyanate, 10 parts by weight of N-methyldiethanolamine, 5 parts by weight of MEK oxime, 3 parts by weight of a bismuth-type carboxylate catalyst, and 2 parts by weight of dimethylformamide, and may be contained in an amount of 10 to wt % based on the total weight of the hair-protective composition.

The sericin may be used without limitation, so long as it is sericin that is commonly used, and may be contained in an amount of 1 to 10 wt % based on the total weight of the hair-protective composition.

In an embodiment, the hair may be synthetic hair or human hair.

In addition, the present disclosure pertains to a method of preparing a hair-protective composition including (a) melting collagen in water, (b) dissolving sericin in the melted collagen and performing filtration, and (c) adding a polyurethane binder thereto and performing stirring.

The preparation method of the present disclosure includes the step of melting collagen in water [i.e. step (a)]. In step (a), the amount of collagen that is contained may be as described above. In step (a), collagen is added to water and melted at about 25 to 35° C. for about 30 min to 2 hr with stirring.

The preparation method of the present disclosure includes the step of dissolving sericin in the melted collagen and performing filtration [i.e. step (b)]. In step (b), the amount of sericin that is contained may be as described above. In step (b), sericin is added thereto and then melted with additional stirring for about 30 min to 2 hr.

The preparation method of the present disclosure includes the step of adding a polyurethane binder thereto and performing stirring [i.e. step (c)]. In step (c), the properties and amount of the polyurethane binder that is contained may be as described above. In step (c), the polyurethane binder is added thereto and stirred for about 30 min to 2 hr, thus obtaining a viscous liquid hair-protective composition, which is referred to as ‘AMICELL’ in the present disclosure.

When AMICELL thus prepared is used, it is very effective at allowing collagen to penetrate to the center of the hair, restoring the surface of damaged hair, reducing the cracking of the hair, increasing elasticity, and reducing the generation of static electricity, and is capable of exhibiting 50% retention of the hair-protective component even after washing 20 to 30 times, and of increasing a moisturized characteristic.

A better understanding of the present disclosure may be obtained through the following example and test example. However, the following example and test example are merely set forth to illustrate the present disclosure, and are not to be construed as limiting the scope of the present disclosure.

### Example

### 1. Preparation of Polyurethane

500 g of polyoxyethylene polyoxypropylene (aqueous modified), 200 g of polyester polyol (aqueous modified) and 100 g of isophorone diisocyanate were mixed for 30 min at 40° C. using a stirrer, after which 100 g of N-methyldiethanolamine, 50 g of MEK (methyl ethyl ketone) oxime, 30 g of a bismuth-type carboxylate catalyst and 20 g of dimethylformamide were further added thereto and mixed at 40° C. for 60 min, thus preparing a polyurethane binder.

### 2. Preparation of AMICELL

Alaska Pollock collagen was prepared as in Korean Patent No. 10-1394230. 200 g of Alaska Pollock collagen was added to 500 g of water, melted with stirring at 30° C. for 1 hr, added with 50 g of sericin, and further melted for 1 hr. The resulting product was filtered to thus remove agglomerated sericin. The resulting product was added with 250 g of the polyurethane binder prepared above and then stirred for 1 hr. The resulting hair-protective composition was a viscous liquid, and was referred to as ‘AMICELL’.

### 3. Use of AMICELL

The AMICELL stock solution thus prepared was mixed and diluted at 1:30 with purified water (pH 7.0 to 7.2, hardness ≤0.7 mol/m³). The wig was immersed in the diluted solution obtained as described above for about 2 to 20 min, and was then dried at 60 to 80° C.

### Test Example

### 1. Hair Image Measurement

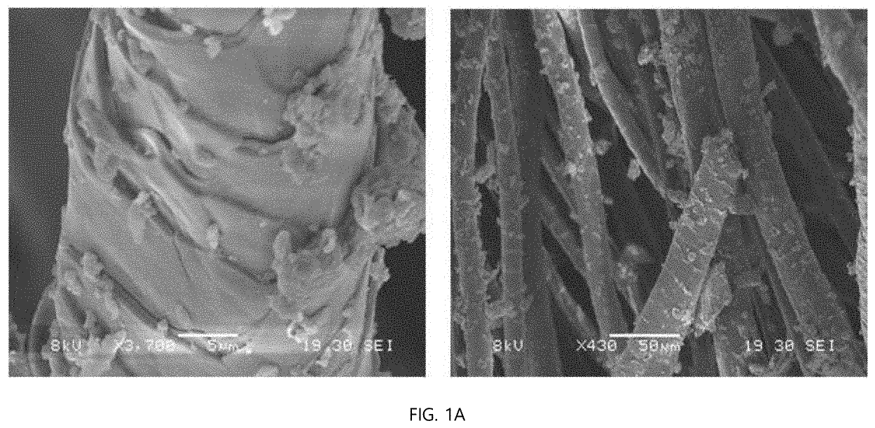

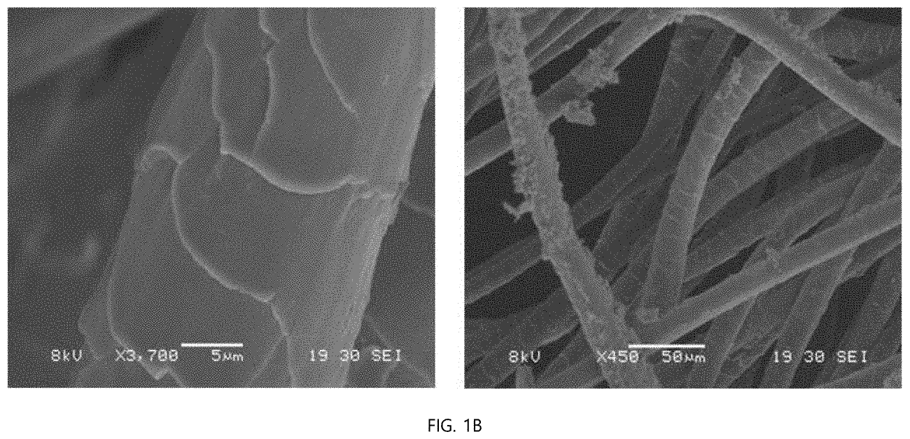

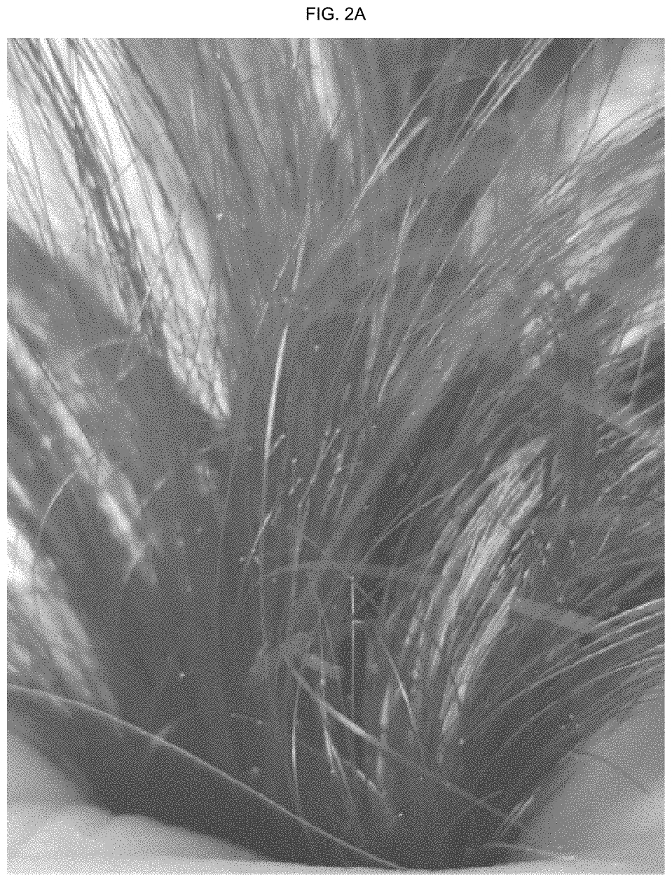

Human hair before and after treatment with AMICELL was observed using a microscope (×3,700 and ×430). Based on the results of observation thereof, as shown in FIGS. 1A and 1B, before using AMICELL, the surface of the hair was damaged, but after using AMICELL, the hair looked like natural hair similar to silk.

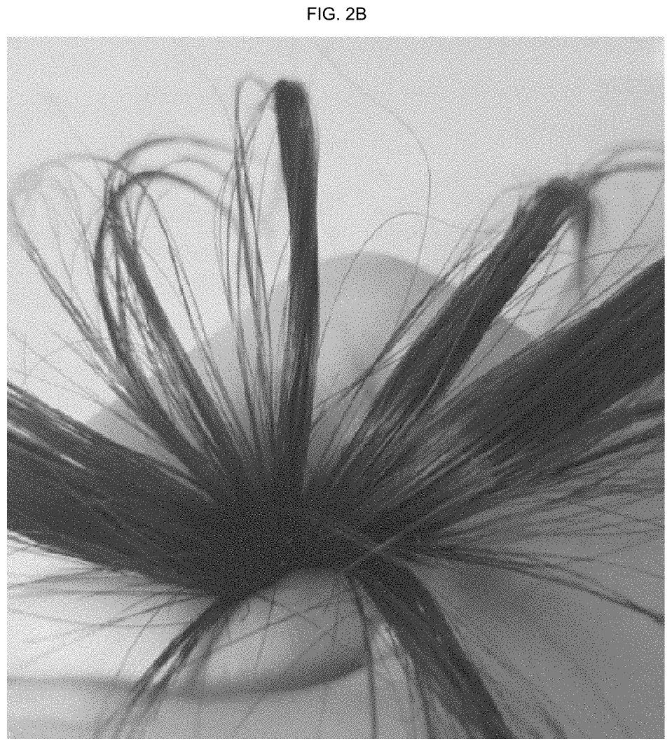

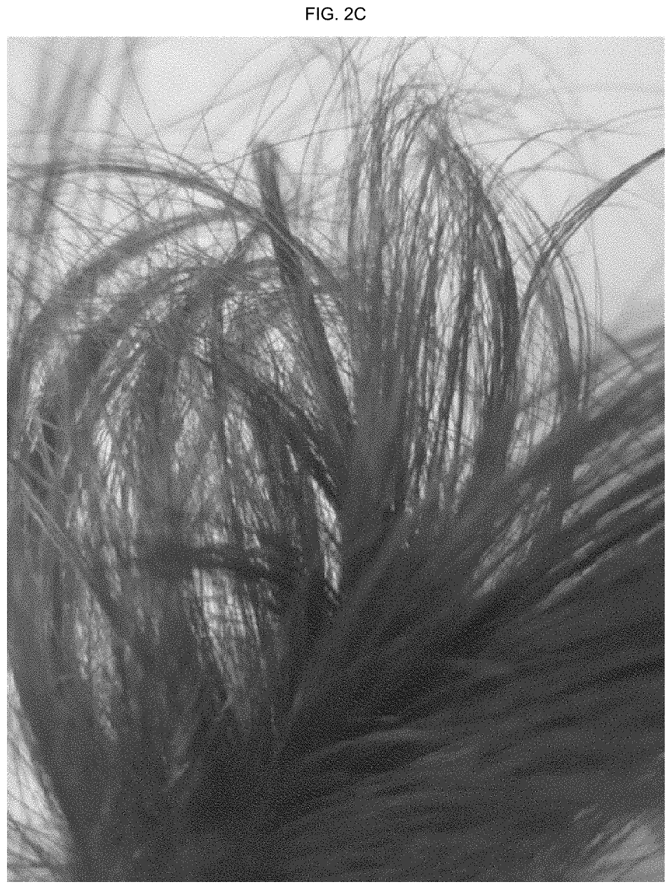

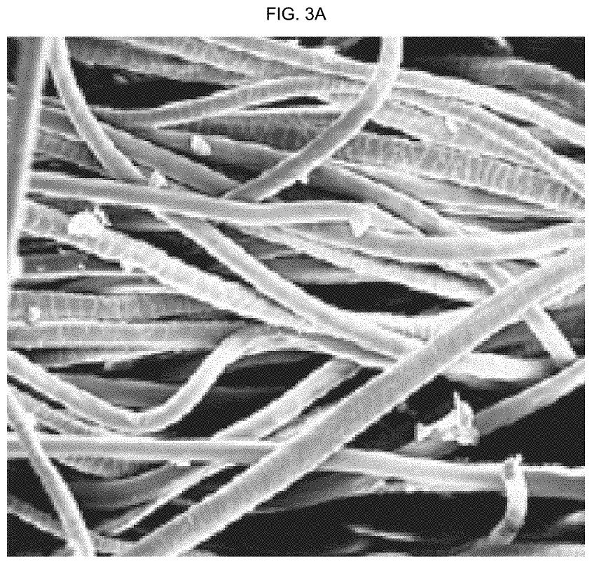

Also, as shown in FIGS. 2A, 2B and 2C, before using AMICELL, the hair was cracked at the ends, was loose, lacked moisture, and showed a weak thin hair shape, but immediately after using AMICELL, the cracking of the hair was alleviated and the hair was coated, showing a strong shape. In addition, cracking of the ends of the hair did not occur again for 1 month after using AMICELL, color fading caused by low moisture was alleviated, and the elasticity of the hair was maintained.

### 2. Penetration Test

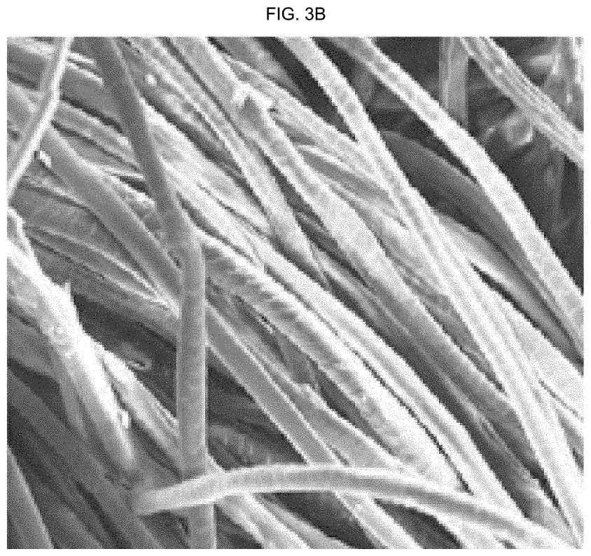

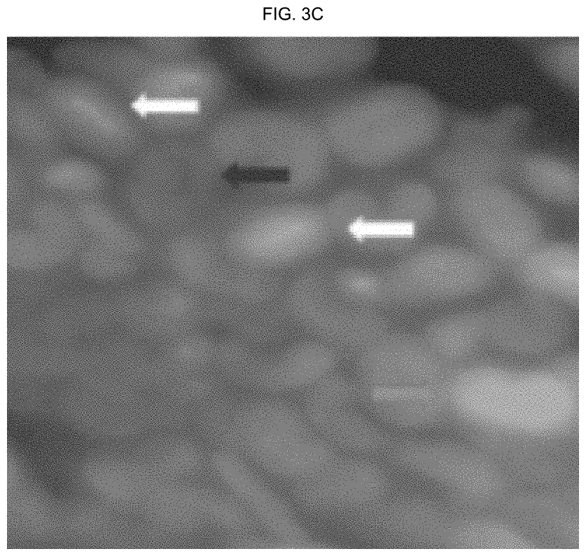

Human hair before and after treatment with AMICELL was observed using a microscope, and the cross section of human hair, after allowing AMICELL mixed with a fluorescent substance to penetrate the hair, was measured through fluorescence. Based on the results of electron microscopy, as shown in FIGS. 3A, 3B and 3C, after treatment with AMICELL [FIG. 3B] compared to before treatment with AMICELL [FIG. 3A], the hair looked neat, and collagen was confirmed to penetrate all the way to the center of the hair [FIG. 3C].

### 3. Antistatic Test

The extent of generation of static electricity was measured before and after treatment of human hair with AMICELL in accordance with the Korean Industrial Standard KS K 0555 (electrostatic test method of fabrics and knitted fabrics). Based on the results thereof, in the case of using silicon, the static electricity of the hair was measured to be about 25,000 V, but when using AMICELL, the static electricity was measured to be 500 to 700 V, indicative of a superior effect of reducing static electricity.

### 4. Washing Test

The hair of a wig was treated with 100% AMICELL and then subjected to machine washing for 8 min (40±3° C., Sereen Dry After AC confirmed), and the amount of AMICELL remaining after washing was measured, and the retention thereof is shown in Table 1 below.

TABLE 1

| | Washing | | | | |
|---|---|---|---|---|---|
| | 1^{st} | 2^{nd} | 3^{rd} | 4^{th} | 5^{th} |
| | washing | washing | washing | washing | washing |
| Retention | 98.3% | 97.8% | 97.0% | 96.3% | 95.2% |

As is apparent from Table 1, it was confirmed that retention was 95% or more after washing five times and 50% after washing 20 to 30 times.

### 5. Moisturizing Test

In accordance with the Korean Industrial Standard KS K ISO6330 (home washing and drying process for textile testing), the moisturized characteristic of human hair was measured before and after treatment with AMICELL. Based on the results thereof, the moisturized characteristic was increased by 400% after treatment with AMICELL.

What is claimed is:

1. A hair-protective composition comprising collagen, a polyurethane binder, sericin and water.

2. The hair-protective composition of claim 1, wherein the collagen is contained in an amount of 10 wt % to 30 wt % based on a total weight of the hair-protective composition.

3. The hair-protective composition of claim 1, wherein the polyurethane binder is prepared by mixing 50 parts by weight of polyoxyethylene polyoxypropylene, 20 parts by weight of polyester polyol, 10 parts by weight of isophorone diisocyanate, 10 parts by weight of N-methyldiethanolamine, 5 parts by weight of MEK (methyl ethyl ketone) oxime, 3 parts by weight of a bismuth-type carboxylate catalyst, and 2 parts by weight of dimethylformamide.

4. The hair-protective composition of claim 1, wherein the polyurethane binder is contained in an amount of 10 wt % to 40 wt % based on a total weight of the hair-protective composition.

5. The hair-protective composition of claim 1, wherein the sericin is contained in an amount of 1 wt % to 10 wt % based on a total weight of the hair-protective composition.

6. The hair-protective composition of claim 1, wherein the hair is synthetic hair or human hair.

7. A method of preparing a hair-protective composition, comprising:
melting collagen in water;
dissolving sericin in the melted collagen and performing filtration; and
performing addition with a polyurethane binder and performing stirring.
